(19) United States
(12) Patent Application Publication
(10) Pub. No.: US 2015/0342956 A1
(43) Pub. Date: Dec. 3, 2015

(54) METHODS OF TREATMENT FOR CONDITIONS MEDIATED BY SUBSTANCE P

(71) Applicant: Beller Pharmaceuticals LLC, Hilton Head, SC (US)

(72) Inventor: Thomas C. Beller, Hilton Head, SC (US)

(21) Appl. No.: 14/729,839

(22) Filed: Jun. 3, 2015

Related U.S. Application Data
(60) Provisional application No. 62/006,988, filed on Jun. 3, 2014.

(51) Int. Cl. A61K 31/5377; A61K 31/197; A61K 31/195; A61K 31/435; A61K 31/4439

(52) U.S. Cl. CPC A61K 31/5377; A61K 31/435; A61K 31/4439; A61K 31/195; A61K 31/197

(57) ABSTRACT

Methods for treating conditions mediated by substance P, including conditions resulting from an inappropriate anti-parasitic immune response, are described.

### RELATED APPLICATIONS

This application claims the benefit of, and priority to, U.S. Provisional Application No. 62/006,988, filed Jun. 3, 2014, which is incorporated herein by reference in its entirety.

### BACKGROUND

A variety of symptoms have traditionally been diagnosed as indicative of allergies, asthma, and/or acid reflux (atypical acid reflux, silent acid reflux, or laryngopharyngeal reflux). As such, they have been associated with mixed treatment strategies having suboptimal benefits. Improved methods of diagnosing and treating patients with these symptoms are needed.

### SUMMARY

The present invention provides, in various embodiments, methods for treating one or more symptoms of a condition mediated by substance P. In various embodiments, the methods comprise administering to a subject in need thereof an effective amount of at least one of immunotherapy, an acid suppressor, a GABA analog, and an NK-1 inhibitor.

In some embodiments, the condition results from an inappropriate anti-parasitic immune response.

In some embodiments, the condition is immunologic neurophylaxis syndrome (INS) and the symptoms comprise at least one of post nasal drip, sinus pressure, cough, sneezing, runny nose, nasal congestion, headache, ear pressure, watery eyes, itchy skin, excessive throat clearing, shortness of breath, itchy eyes, nausea, urinary urgency, bedwetting, fatigue, malaise, sore throat, nose bleeds, hoarse voice, itchy throat, palate, or pharynx, pain in the teeth, diminished sense of smell, diminished sense of taste, interstitial cystitis, vulvodynia, glossodynia, pruritus ani, tinnitus, vertigo, imbalance, puffy eyelids, allergic shiners, excessive ear wax production, diarrhea, constipation, acne, ulcerative colitis, and chronic prostatitis.

In some embodiments, the symptoms comprise at least one of sinus pressure, post nasal drip, sneezing, ear pressure, throat clearing, shortness of breath, itchy eyes, watery eyes, and runny nose.

In some embodiments, at least one of the symptoms is associated with allergy or allergic rhinitis.

In some embodiments, at least one of the symptoms is associated with atypical acid reflux or laryngopharyngeal reflux.

In some embodiments, the itchy skin is associated with atopic dermatitis.

In some embodiments, the cough is associated with acid reflux.

In some embodiments, at least one of the diarrhea and the constipation is associated with irritable bowel syndrome.

In some embodiments, the condition is anaphylaxis.

In some embodiments, the condition is infertility.

In some embodiments, the NK-1 inhibitor is aprepitant.

In some embodiments, the NK-1 inhibitor is rolapitant.

In some embodiments, the acid suppressor is omeprazole.

In some embodiments, the GABA analog is gabapentin.

In some embodiments, the GABA analog is R-baclofen.

Additional features and advantages of the present invention are described further below. This summary section is meant merely to illustrate certain features of embodiments of the invention, and is not meant to limit the scope of the invention in any way. The failure to discuss a specific feature or embodiment of the invention, or the inclusion of one or more features in this summary section, should not be construed to limit the invention as claimed.

### BRIEF DESCRIPTION OF THE DRAWINGS

The foregoing summary, as well as the following detailed description of the preferred embodiments of the application, will be better understood when read in conjunction with the appended drawings. For the purposes of illustrating the systems and methods of the present application, there are shown in the drawings exemplary embodiments. It should be understood, however, that the application is not limited to the precise arrangements and instrumentalities shown. In the drawings:

FIG. 1 shows an exemplary schematic of physiological response to parasites or allergens (false parasites) according to some embodiments of the invention. Increased stomach acid activates nerves, such as the vagus nerve, and stimulates release of neurokinins, such as substance P.

FIG. 2 shows another exemplary schematic of physiological response to allergens (false parasites) according to some embodiments. Allergens (false parasites) increase sensitivity of nociceptors and stimulate production of stomach acid through the histamine H₂ receptor, with various effects throughout the body.

FIG. 3 is a bar graph illustrating the relationship between number of oral medications and immunotherapy status in one example.

### DETAILED DESCRIPTION

Embodiments of the present invention relate to novel methods of diagnosing and treating patients having certain symptoms that are inconsistently and/or unsatisfactorily treated in the current clinical environment.

Certain embodiments of the present invention identify a predetermined set of different symptoms as constituting a unified syndrome, designated herein as “immunologic neurophylaxis syndrome” (INS). The symptoms identified as part of INS include: post nasal drip, sinus pressure (often perceived as sinus infections/recurring sinusitus; can be persistent or intermittent), cough, sneezing (especially occurring in fits involving several sneezes occurring in succession), runny nose, nasal congestion, headache (especially frontal headache), ear pressure, watery eyes (excessive tearing; may be associated with dry eye syndrome or not), itchy skin (can be present as isolated itching, prurigo nodularis, or neurodermatitis; may be associated with atopic dermatitis), excessive throat clearing, shortness of breath (sometimes associated with chest tightness; may be mistaken for asthma), itchy eyes, nausea, urinary urgency (pollakiuria), bedwetting, fatigue, malaise, sore throat, nose bleeds (epistaxis), hoarse voice, itchy throat, palate, or pharynx, pain in the teeth (often an extension of sinus pain/pressure), diminished sense of smell and/or taste, interstitial cystitis (bladder pain syndrome), vulvodynia (burning vaginal syndrome), glossodynia (burning mouth syndrome), pruritus ani (itching around the anus), tinnitus, vertigo, a sense of imbalance (without true vertigo), puffy eyelids, allergic shiners (dark circles under the eyes), and excessive ear wax production. Additional symptoms that may be treated as part of INS include irritable bowel syndrome (can present with frequent diarrhea, often right after eating, and sometimes with constipation; can alternate between the two), acne, ulcerative colitis, and chronic prostatitis.

INS is believed to have its basis in an anti-parasitic immune response coming through the nervous system.

In the context of improved attention to clean food, clean water, and waste removal, industrialized countries have limited their exposure to microbes, parasites, and disease-causing bacteria. Concomitantly, there has been a significant increase in the incidence of chronic inflammatory disorders (e.g., allergic and autoimmune disorders). Several epidemiologic studies have described an evolutionary link or “evolved dependence” between the increased incidence of allergy and the more limited exposure to microbes, parasites, and other “old friends.” Rather than provoking aggressive immune responses, these no-longer-present organisms have been found to be associated with a pattern of maturation of dendritic cells and regulatory T cells helping to control mediators of inflammation.

The inventor has identified that immunoglobulin E (IgE)-mediated responses are no longer limited to parasites; they are activated by allergens, such as pollens, dust mites, pets, trees, stinging insects, and/or selected foods. The body attacks the allergen as it would attack a parasite; the immune response is the same. Thus, allergy may be understood as an “anti-parasitic mistake.”

Without wishing to be bound to theory (and without limiting the scope of the invention claimed), it is believed that INS is caused by an inappropriate and exaggerated fending-off type of response of the nervous system, designed to protect against parasitic invasion. It is a state of disease, or syndrome, because it is inappropriate in a setting where parasites are not present. INS can occur in a setting of allergy and/or acid reflux (silent or otherwise), but does not require either to be present.

The proposed mechanism of the anti-parasitic immune response underlying INS is not to be construed as limiting in any way. However, it is believed that the response involves a process comprising:

(1) Allergy.

In some cases, allergy can be an underlying force of INS and may initiate the anti-parasitic immune response. The body detects an allergen and mistakes it for a parasite. The body typically reacts to the allergen (false parasite) with an IgE-mediated response.

(2) Acid Reflux.

Histamine receptor activity in the stomach and acid production, which are involved in the pathways of defense against pathogens, especially parasites, are activated. Histamine release, which is associated with IgE-mediated responses, stimulates acid production in the stomach via histamine H₂ receptors (with a normal pH ranging from about 1.5 to 3.5 and increased acidic values associated with the intake of food). Stomach acid, which is known to be anti-microbial, is identified in the present invention as anti-parasitic in its main function, and increased stomach acid is believed to be at the center of the anti-parasitic immune response described herein.

(3) Vagus Nerve Activation.

Stomach acid is detected in the lower esophagus by the nervous system, which has far-reaching interaction with other tissues of the body including, but not limited to, those containing mucosal tissue or skin. The neurogenic component associated with acid production in the stomach involves the vagus nerve, which provides both motor and sensory input to the laryngopharangeal area, sinuses, periorbital/frontal head, and ears. Vagal innervation (e.g., ventral vagal innervation of the larynx and esophagus) may be amplified in response to increased acid production, stress, and/or allergen exposure. Other nerves that communicate with the vagus nerve may also be activated (e.g., other cranial nerves, pelvic splanchnic nerves, etc.), which can also release substance P and contribute to symptoms of INS.

(4) Substance P Release.

In addition to the sensory impact, innervation of the vagus nerve and other connecting nerves is also associated with release of neurokinins, including substance P. The release of substance P, a potent mediator of inflammation, may cause pain, swelling, congestion, and/or increased mucous production. The resulting symptoms include those identified above as part of INS.

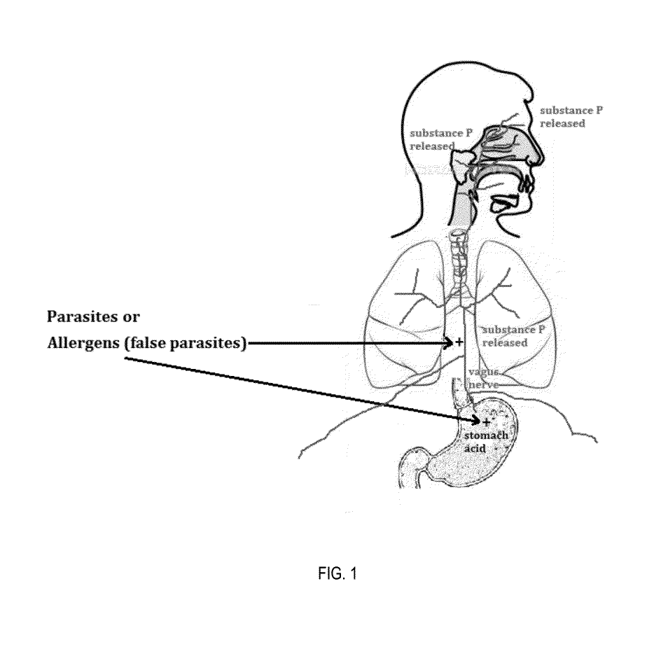

As shown in FIG. 1, an increase in stomach acid may be a central part of the physiological response to exposure to parasites or allergens (false parasites). Nerves in the body, such as the vagus nerve, detect stomach acidity and can be stimulated in this setting to release neurokinins, such as the neuromediator substance P, into diffuse tissues throughout the body. Due to confusion in the body (mistaking allergens for parasites), allergies are more common and the vagus nerve, and other connected nerves, are more active.

Substance P is identified herein as having anti-parasitic actions that result in the symptoms of INS, including, but not limited to, sinus pressure, post nasal drip, sneezing, ear pressure, throat clearing, shortness of breath, itchy eyes, watery eyes, and runny nose, none of which are known to be associated with substance P.

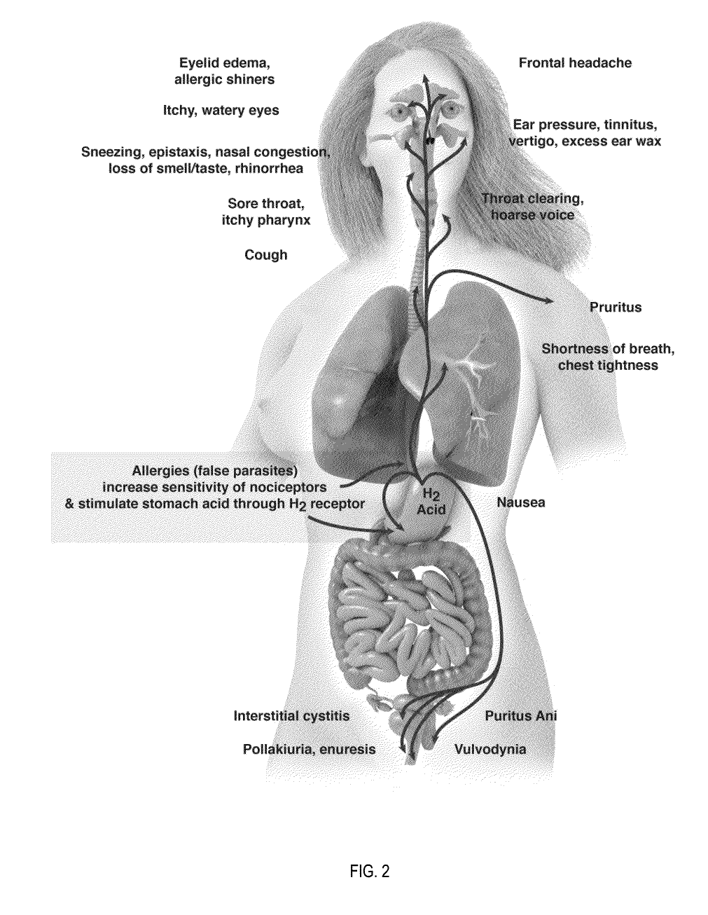

FIG. 2 illustrates some of the effects that the anti-parasitic immune response described herein can have throughout the body.

In some cases, chronic exposure to parasites or allergens may increase the vagus nerve's sensitivity to stomach acid. Sensory nerve fibers may be hyperactive, releasing neurokinins and causing INS to develop even when there are normal levels of stomach acid.

In some cases, with INS, an allergen, such as pollen, may contribute to symptoms, but not through an antibody response (e.g., by stimulating toll-like receptors which activate the reflux pathway).

Allergy and/or acid reflux may be involved in the amplified vagal innervation and subsequent release of substance P associated with the anti-parasitic immune response described above. Accordingly, in various embodiments of the invention, INS may be treated, for example, using immunotherapy (allergy shots) and/or acid suppression (e.g., high dose acid suppression using a proton pump inhibitor such as omeprazole, a histamine H₂-receptor antagonist such as famotidine or ranitidine, etc.). Prior to the present invention it would not have been obvious to one of ordinary skill in the art, for example, to treat a symptom of INS, such as itchy eyes, with omeprazole (brand name Prilosec®). Immunotherapy dosing is according to guidelines published by the American Academy of Allergy, Asthma, and Immunology. Dosing of the oral medications may vary by patient, but typical dosing of omeprazole is typically 20 mg twice daily for patients under 12 years of age, and 40 mg twice daily for patients over 12 years of age. Famotidine (brand name Pepcid®) and ranitidine (brand name Zantac®) are typically used for infants and young children at doses of about 75-150 twice daily.

In some embodiments, INS may be treated, for example, using a gamma-aminobutyric acid (GABA) analog, such as gabapentin (which blocks the release of substance P; originally developed as a seizure medicine) or baclofen (GABA_{B} receptor agonist). When baclofen is used, the R enantiomer is preferred. Dosing of these medications may vary by patient. Typical dosing of baclofen may range from 5-40 mg twice daily, with 20 mg twice daily being the most common dose. Typical dosing of gabapentin may range from about 100-7200 mg per day, typically ranging from 300 mg once a day to 300 mg three times a day.

In certain embodiments, a neurokinin 1 (NK-1) receptor inhibitor (substance P antagonist) may be used to treat INS. NK-1, a G protein-coupled receptor (GPCR), is the primary receptor for substance P, which is a neuropeptide found naturally in nerve endings throughout the body. An exemplary NK-1 inhibitor/substance P antagonist is aprepitant (brand name Emend®; available in 40 mg, 80 mg, and 125 mg capsules). Other NK-1 hereinafter developed and existing inhibitors (e.g., long-acting rolapitant) are also known to those of ordinary skill in the art, and may be used to treat INS and other conditions associated with the anti-parasitic immune response, according to various embodiments of invention.

In some embodiments, the above-described treatments for INS can reduce the unnecessary use of oral steroids, asthma medications, antibiotic therapies, and/or surgery.

In further embodiments, NK-1 inhibitors may be used, for example, to treat anaphylaxis. The proposed mechanism is not to be construed as limiting in any way; however, it is believed that substance P, which is known to be a significant vasodilator, is a contributing factor to anaphylaxis, and may act as an important mediator of the drop in blood pressure (hypotension) that is observed with anaphylactic shock. Thus, administration of an NK-1 inhibitor/substance P antagonist can block anaphylactic shock.

In additional embodiments, NK-1 inhibitors may be used, for example, to treat infertility. The proposed mechanism is not to be construed as limiting in any way; however, it is believed that mucosal tissue is a common area for substance P release due to its anti-parasitic nature and that a fetus may be understood in essence as a parasite. Accordingly, substance P is believed to prevent implantation and/or otherwise reduce fetus viability. Administration of an NK-1 inhibitor/substance P antagonist can block these effects and help women become pregnant.

### EXAMPLES

### Example 1

Some symptoms of INS have previously been associated with atypical reflux. This example describes a retrospective medical record review for cases in which these symptoms were treated with immunotherapy (allergy shots) and/or oral medications (omeprazole, gabapentin, and/or baclofen).

The population for this example included patients 18 years or older with a diagnosis of atypical reflux and a recorded diagnostic code of 530.81 (see, e.g., http://www.cms.gov/Medicare/Coding/ICD9ProviderDiagnosticCodes/index.html). A randomized sample of 307 cases was derived from a total of 650 patients.

The 14 most frequently-occurring INS symptoms were determined to be (in decreasing order of observed frequency, from >75% to about 34%): post nasal drip, sinus pressure, rhinorrhea, dry cough, throat clearing, itchy eyes, sneezing, frontal headache, ear pressure, watery eyes, hoarse voice, shortness of breath, fatigue, and itchy skin.

Of the 307 cases, 191 (62.2%) patients tested positive for allergy, 74 (24.1%) were negative, and 42 (13.7%) had unknown allergy status. Allergy refers to IgE-mediated allergies such as ragweed, dust mites, trees, grass, pets, stinging insects, peanuts, etc. Of the 191 cases with allergy, 57 (29.8%) had no immunotherapy, 8 (4.2%) had received immunotherapy in the past, and 126 (66.0%) were currently receiving immunotherapy. Of the 126 currently receiving immunotherapy (abbreviated below as “IT”), 70 (55.6%) had reached maintenance dosing (defined as the effective, therapeutic dose; “on main”) and 56 (18.2%) had not yet reached maintenance dosing (“not on main”). The period of immunotherapy is about 3-5 years, and individuals typically reach maintenance dosing by the second half of the first year.

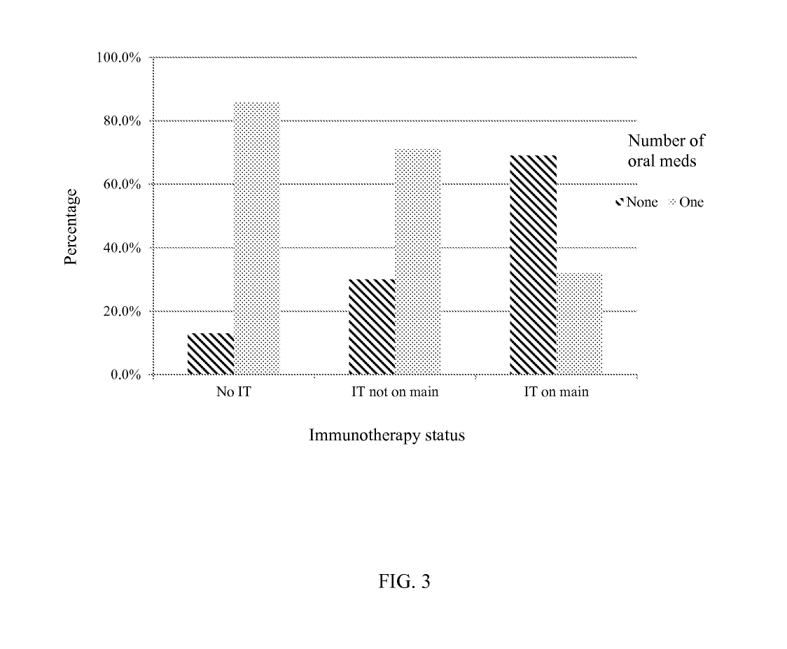

Of the 307 cases, 213 (69.3%) were taking one medication and 86 (28%) were taking no medication. Seven patients were taking two medications, and one patient was taking three medications. Three oral medications used to treat INS were compared: high dose acid suppression using omeprazole (40 mg twice a day), baclofen (40 mg twice a day), and gabapentin (300 mg gradually increasing from one to three times a day). The bar graph of FIG. 3 shows the relationship between number of oral medications and immunotherapy status.

Global level of improvement (GLI) was represented by a 0-100% score. At each clinical visit, patients were asked to estimate the percentage of their overall improvement. Those figures were entered into the medical record as a GLI score. Trials documented as failed (stopped either due to lack of benefit or dose-limiting side effects) or inadequate (dose never titrated to benefit or side effect) are not included in the results below.

Table 1 shows the medication-associated GLI scores for patients who were not on immunotherapy (no allergy, or positive allergy but no immunotherapy).

TABLE 1
No IT

| Oral medication | Mean GLI | Standard Deviation | N |
|---|---|---|---|
| None | 8.89 | 26.67 | 9 |
| Gabapentin | 65.58 | 31.85 | 73 |
| Baclofen | 53.94 | 46.51 | 4 |
| Omeprazole | 56.67 | 34.79 | 12 |

Table 2 shows the medication-associated GLI scores for patients who were on immunotherapy but had not yet reached maintenance dosing. The maintenance does is defined as the maximum dose planned for that patient. Patients are maintained on shots for a number of years at a dose that no longer increases.

TABLE 2
IT not on maintenance dosing

| Oral medication | Mean GLI | Standard Deviation | N |
|---|---|---|---|
| None | 69.55 | 28.85 | 11 |
| Gabapentin | 69.09 | 25.43 | 22 |
| Baclofen | 88.33 | 16.07 | 3 |
| Omeprazole | 60.00 | 27.04 | 9 |

Table 3 shows the medication-associated GLI scores for patients who were on immunotherapy and had reached maintenance dosing.

TABLE 3
IT on maintenance dosing

| Oral medication | Mean GLI | Standard Deviation | N |
|---|---|---|---|
| None | 86.86 | 11.70 | 35 |
| Gabapentin | 70.29 | 27.81 | 17 |
| Baclofen | 70.00 | — | 2 |
| Omeprazole | 80.00 | — | 1 |

Table 4 shows the medication-associated GLI scores for patients without allergy.

TABLE 4
No allergy

| Oral medication | Mean GLI | Standard Deviation | N |
|---|---|---|---|
| None | 13.33 | 32.66 | 6 |
| Gabapentin | 55.19 | 34.86 | 36 |
| Baclofen | 65.00 | 49.50 | 2 |
| Omeprazole | 41.00 | 45.88 | 5 |

This study indicated the likelihood of allergy when INS symptoms are observed, and patients on immunotherapy generally reported greater improvement than those not on immunotherapy, regardless of maintenance dosing. For patients who were not on immunotherapy (with or without allergy), improved GLI was observed for each of the three oral medications, compared to no medication, indicating that acid suppression therapy and/or treatment with a drug that reduces release of substance P, neither of which has previously been described in guidelines for managing atypical reflux, can be useful for treating INS based on the mechanism of the anti-parasitic immune response described above.

These results are detailed in Coggins, C. C. Allergy and Immunology: An Organizing Paradigm for Atypical Reflux. Dissertation, Loyola University of New Orleans, School of Nursing, which is incorporated by reference herein in its entirety.

### Example 2

This example describes three exemplary cases of INS. Patients were given a list of INS symptoms and asked to rank those present on a scale of 1-10, with 10 indicating most severe. Treatment was provided (gabapentin, omeprazole, and/or immunotherapy). Patients were then asked to screen the INS symptoms again (with 0 indicating that the symptom was not present), and also to provide a GLI score.

Example Case #1.

Patient A.C. is a 14-year-old female who presented complaining of cough, runny nose, and sneezing. Further questions revealed multiple symptoms of INS, including those noted below. She was started on gabapentin (900 mg) and returned in two weeks for re-evaluation. She reported 60% global level of improvement with gabapentin. She was also started on immunotherapy for its long-lasting benefits. She returned for further evaluation three months later to report she had improved by 85% and had stopped the gabapentin more than a month prior to her last visit. Her symptoms were scored for severity from 1 (mild) to 10 (severe) on initial evaluation, after gabapentin, and after three months of immunotherapy. Her scores are below:

| | Initial Visit | Gabapentin | Immunotherapy |
|---|---|---|---|
| Cough | 9 | 2 | 2 |
| Post Nasal Drip | 8 | 1 | 0 |
| Repetitive Sneezing | 10 | 2 | 0 |
| Sinus Pressure | 10 | 3 | 0 |
| Throat Clearing | 7 | 1 | 0 |
| Shortness of Breath | 6 | 4 | 0 |
| Chest Tightness | 7 | 2 | 0 |
| Frontal Headache | 10 | 1 | 0 |
| Ear Pressure | 5 | 2 | 0 |
| Itchy Eyes | 10 | 1 | 2 |
| Watery Eyes | 5 | 2 | 0 |
| Runny Nose | 7 | 1 | 0 |
| Sore Throat | 7 | 1 | 0 |
| Fatigue | 6 | 3 | 0 |
| Malaise | 5 | 1 | 0 |
| Itchy Skin | 10 | 4 | 0 |
| Glossodynia | 7 | 3 | 0 |
| Vertigo | 7 | 1 | 0 |
| Puffy Eyelids | 8 | 3 | 0 |
| Hoarse Voice | 2 | 1 | 0 |
| Itchy Pharynx | 10 | 3 | 0 |
| Pain in the teeth | 3 | 1 | 0 |
| Global Improvement | — | 60% | 85% |

Example Case #2.

Patient C.F. is a 72-year-old male who presented complaining of cough, post nasal drip, and runny nose. Further questions revealed multiple symptoms of INS. The patient was started on immunotherapy and returned for follow-up after four months of immunotherapy. Due to his busy schedule the patient had missed several shot visits and his immunotherapy doses were never increased to the maximum, maintenance dose. He was receiving doses at 1/10^{th} of the maintenance dose. He reported 40% improvement. Omeprazole 40 mg two times daily was added to his regimen and he returned for re-evaluation one week later to report 100% improvement. His symptom scores are below:

| | | Sub-Optimal | |
|---|---|---|---|
| | Initial Visit | Immunotherapy | Omeprazole |
| Cough | 10 | 6 | 0 |
| Post Nasal Drip | 10 | 7 | 0 |
| Repetitive Sneezing | 5 | 3 | 0 |
| Nasal Congestion | 10 | 5 | 0 |
| Sinus Pressure | 10 | 6 | 0 |
| Throat Clearing | 10 | 7 | 0 |
| Chest Tightness | 10 | 5 | 0 |
| Frontal Headache | 6 | 4 | 0 |
| Ear Pressure | 5 | 2 | 0 |
| Itchy Eyes | 7 | 5 | 1 |
| Watery Eyes | 7 | 5 | 0 |
| Runny Nose | 10 | 5 | 0 |
| Sore Throat | 7 | 5 | 0 |
| Epistaxis | 6 | 4 | 0 |
| Vertigo | 10 | 4 | 0 |
| Pollakiuria | 6 | 3 | 0 |
| Hoarse Voice | 7 | 3 | 1 |
| Pain in the teeth | 5 | 5 | 0 |
| Excessive Ear Wax | 5 | 4 | 1 |
| Global Improvement | — | 40% | 100% |

Example Case #3.

Patient A.B. is a 9-year-old girl who presented with her mother complaining of shortness of breath and cough. Further questions revealed multiple symptoms of INS including severe bedwetting. Her mother stated that she commonly wets the bed 2-3 times each night. She was started on omeprazole 20 mg twice a day and returned in a week. She had significant improvement in many symptoms including dramatic benefits in bedwetting (none since starting omeprazole), but was having some headaches as a possible side effect of omeprazole. Her mother estimated 45% global level of improvement. The omeprazole was discontinued due to headaches and she was started on gabapentin. Her mother was advised to increase the dose slowly until it seemed to help or was causing side effects. She returned in two weeks for re-evaluation and had achieved 900 mg twice daily on gabapentin. Her mother estimated 85% global level of improvement.

| | Initial Visit | Omeprazole | Gabapentin |
|---|---|---|---|
| Cough | 8 | 4 | 3 |
| Post Nasal Drip | 8 | 3 | 3 |
| Throat Clearing | 8 | 8 | 5 |
| Chest Tightness | 6 | 3 | 0 |
| Frontal Headache | 0 | 8 | 0 |
| Shortness of breath | 5 | 5 | 5 |
| Watery Eyes | 6 | 2 | 0 |
| Runny Nose | 10 | 5 | 0 |
| Sore Throat | 7 | 5 | 0 |
| Fatigue | 4 | 2 | 2 |
| Malaise | 4 | 2 | 2 |
| Pollakiuria | 8 | 2 | 2 |
| Vulvodynia | 3 | 0 | 0 |
| Bedwetting | 10 | 0 | 0 |
| Nausea | 4 | 0 | 0 |
| Global Improvement | — | 45% | 85% |

As will be understood by those skilled in the art having read the foregoing disclosure, an effective dose is identified by titrating up until benefits are seen without significant side effects.

While there have been shown and described fundamental novel features of the invention as applied to the preferred and exemplary embodiments thereof, it will be understood that omissions and substitutions and changes in the form and details of the disclosed invention may be made by those skilled in the art without departing from the spirit of the invention. Moreover, as is readily apparent, numerous modifications and changes may readily occur to those skilled in the art. Hence, it is not desired to limit the invention to the exact construction and operation shown and described and, accordingly, all suitable modification equivalents may be resorted to falling within the scope of the invention as claimed.

What is claimed is:

1. A method for treating one or more symptoms of a condition mediated by substance P, comprising administering to a subject in need thereof an effective amount of at least one of immunotherapy, an acid suppressor, a GABA analog, and an NK-1 inhibitor.

2. The method of claim 1, wherein the condition results from an inappropriate anti-parasitic immune response.

3. The method of claim 1, wherein the condition is immunologic neurophylaxis syndrome (INS) and the symptoms comprise at least one of post nasal drip, sinus pressure, cough, sneezing, runny nose, nasal congestion, headache, ear pressure, watery eyes, itchy skin, excessive throat clearing, shortness of breath, itchy eyes, nausea, urinary urgency, bedwetting, fatigue, malaise, sore throat, nose bleeds, hoarse voice, itchy throat, palate, or pharynx, pain in the teeth, diminished sense of smell, diminished sense of taste, interstitial cystitis, vulvodynia, glossodynia, pruritus ani, tinnitus, vertigo, imbalance, puffy eyelids, allergic shiners, excessive ear wax production, diarrhea, constipation, acne, ulcerative colitis, and chronic prostatitis.

4. The method of claim 3, wherein the symptoms comprise at least one of sinus pressure, post nasal drip, sneezing, ear pressure, throat clearing, shortness of breath, itchy eyes, watery eyes, and runny nose.

5. The method of claim 3, wherein at least one of the symptoms is associated with allergy or allergic rhinitis.

6. The method of claim 3, wherein at least one of the symptoms is associated with atypical acid reflux or laryngopharyngeal reflux.

7. The method of claim 3, wherein the itchy skin is associated with atopic dermatitis.

8. The method of claim 3, wherein the cough is associated with acid reflux.

9. The method of claim 3, wherein at least one of the diarrhea and the constipation is associated with irritable bowel syndrome.

10. The method of claim 1, wherein the condition is anaphylaxis.

11. The method of claim 1, wherein the condition is infertility.

12. The method of claim 1, wherein the NK-1 inhibitor is aprepitant.

13. The method of claim 1, wherein the NK-1 inhibitor is rolapitant.

14. The method of claim 1, wherein the acid suppressor is omeprazole.

15. The method of claim 1, wherein the GABA analog is gabapentin.

16. The method of claim 1, wherein the GABA analog is R-baclofen.
